(19) United States
(12) Patent Application Publication
(10) Pub. No.: US 2020/0000775 A1
(43) Pub. Date: Jan. 2, 2020

(54) ATTENUATION OF NEUROPATHIC PAIN AFTER SPINAL CORD INJURY

(71) Applicant: University of Kentucky Research Foundation, Lexington, KY (US)

(72) Inventors: Chen Guang Yu, Lexington, KY (US); James W. Geddes, Lexington, KY (US)

(21) Appl. No.: 16/459,302

(22) Filed: Jul. 1, 2019

Related U.S. Application Data
(60) Provisional application No. 62/691,969, filed on Jun. 29, 2018.

(51) Int. Cl. A61K 31/4184; C12N 9/10

(52) U.S. Cl. CPC A61K 31/4184; C12Y 203/01; C12N 9/1029

(57) ABSTRACT

Methods for treating neuropathic pain caused by a traumatic spinal cord injury are described. A method comprising administering an effective amount of flubendazole, an α-tubulin acetylation inhibitor, an endosomal NR1 and pERK1/2 inhibitor, a mitochondrial cyclin b1 inhibitor, a microtubule destabilizing drug, or combinations thereof to the patient suffering from the traumatic spinal cord injury. Also described is a method for preventing neuropathic pain in a patient with a spinal cord injury at risk for developing neuropathic pain comprising administering administrating an effective amount of flubendazole to a patient with the spinal cord injury at risk of developing neuropathic pain.

### RELATED APPLICATIONS

This application is related to U.S. Provisional Application Ser. No. 62/691,969 filed Jun. 29, 2018, the entire disclosure of which is incorporated herein by this reference.

### GOVERNMENT INTEREST

This invention was made with government support under grant number UL1TR000117 awarded by the National Institutes of Health. The government has certain rights in the invention.

### TECHNICAL FIELD

The present invention relates to a method of using flubendazole and related compounds for the treatment of pain.

### BACKGROUND AND SUMMARY

Neuropathic pain is a debilitating consequence of spinal cord injury (SCI) that remains difficult to treat. Microtubule hyper-stabilization and α-tubulin acetylation (a marker for microtubule hyper-stabilization) are involved in pain transmission [1, 2]. However, the anti- nociceptive effects of microtubule destabilization in neuropathic pain have not been previously investigated following spinal cord injury (SCI). Microtubule-destabilizing agent Flubendazole (FluBZ) has been widely used in the treatment of intestinal and neural parasites in human. Here, we show a novel anti-nociceptive effect of FluBZ on intracellular pERK1/2 pain signal transduction and spontaneous pain behaviors after excitotoxic SCI. The excitotoxic SCI was produced by intraspinal microinjection of AMPA/metabotropic receptor agonist quisqualic acid (QUIS). Intraperitoneal (IP) injection with 10 mg/kg/day (n=10) of FluBZ to Sprague-Dawley rats for 1 week was administered 3 hrs post-excitotoxic injury between T12 and L1. Pain behavioral assessments demonstrated that this FluBZ treatment resulted in a significant delay in the onset of painful grooming behaviors, reduction in size of the painful grooming area, and decreased severity of the painful grooming severity after excitotoxic SCI in rats, compared to vehicle-treated controls (n=10). FluBZ IP treatment also reduced the incidence of the pain behaviors following excitotoxic SCI. Mechanistic studies revealed that FluBZ IP treatment significantly reduced the excitotoxic injury-induced upregulation of α-tubulin acetylation, acetyltransferase MEC-17 activity, endosomal NR1 activity, endosomal pERK1/2 activation, and mitochondrial cyclin B1 activity in the dorsal horn of rat spinal cord after excitotoxic injury. In conclusion, our results suggested that proper microtubule destabilization by FluBZ IP administration attenuated spontaneous pain behaviors through inhibiting microtubule hyper-stabilization, α-tubulin acetylation, α-tubulin acetyltransferase ATAT1/MEC-17, endosomal NR1/pERK1/2, and mitochondrial cyclin b1 signaling cascade after excitotoxic SCI.

Neuropathic pain is one of the most common and devastating consequences of traumatic spinal cord injury (SCI). Recent data suggest that about 80% of patients experience SCI-pain, which is primarily spontaneous, chronic, and refractory to many conventional analgesic treatments [3-7]. The SCI-pain has been significantly associated with depression and chronic fatigue and has a greater negative impact on their quality of life of SCI patients. Currently there is no effective FDA-approved non-opioid analgesic treatment available and the mechanisms underlying SCI-neuropathic pain is still poorly understood [6]. Opioid use is limited because prescription opioid crisis is a critical public health issue (https://www.drugabuse.gov/drugs-abuse/opioids/opioid-overdose-crisis). After SCI, there is an urgent need for further research to better understand pain mechanisms and develop therapeutics to prevent and reduce chronic neuropathic pain.

It is well documented that excitotoxic injury results in spinal dorsal horn activation of extracellular signal-regulated kinases 1/2 (pERK1/2, a key pain marker [8-10]) that play important role in pain transmission. Intraspinal injection of the AMPA- and metabotropic receptor agonist quisqualic acid (QUIS) has been shown to mimic the pathological sequella of activation of glutamate receptors, ERK1/2 activation, and spontaneous neuropathic pain behaviors observed in contusive or ischemic injured spinal cords following spinal cord injury [10-13]. The spontaneous pain behaviors are progressive self-injurious overgrooming behaviors of the affected at- and below-level dermatome and are thought to resemble painful dysesthesia observed in in SCI patients. The specific intracellular glutamatergic cascades produce maladaptive synaptic circuits and neuronal hyperexcitability that result in enhanced pain transmission in the spinal dorsal horn after SCI [14]. The pERK1/2 signaling and excitotoxic pain animal model are widely used for evaluation of pharmacological interventions. However, improved treatments of this SCI-neuropathic pain conditions require a thorough understanding of the mechanism of intracellular pain signal transduction and translocation underlying the pain transmission after SCI.

Microtubule hyper-stabilization and α-tubulin acetylation (a marker for microtubule hyper-stabilization) are involved in pain transmission [1, 2], but the anti-nociceptive effects of microtubule destabilization in neuropathic pain have not been previously investigated following spinal cord injury (SCI). Microtubules are highly dynamic filaments assembled from αβ-tubulin heterodimers and play important roles in intracellular transport. During microtubule dynamic, microtubules switch stochastically between stabilizing (from depolymerization to polymerization) and destabilizing phases (from polymerization to depolymerization) [15]. Hyper-stable microtubules, α-tubulin acetylation (a marker for microtubule hyper-stabilization), and acetyltransferase Atat1/MEC-17 are involved in abnormal intracellular molecule and organelle transport, inflammasome activation, and pain transmission [1, 16, 17]. Flubendazole (FluBZ, an approved anthelmintic drug) is known to be a mild microtubule destabilization drug that inhibits microtubule polymerization by binding to tubulin, regulates microtubule dynamics, and inhibits abnormal microtubule-based intracellular molecule and organelle transport. The purpose of the present study was to examine whether microtubule destabilizing drug FluBZ would reduce intracellular glutamatergic pain signal transduction and prevent/reduce neuropathic pain after SCI in the rat excitotoxic SCI pain model.

Sustained activation of glutamate receptor-pERK1/2 pathway [9, 18, 36], B cell autoimmunity [19, 35-40], and astroglial responses [41-44] are major contributors to chronic pain [45-50] and traumatic SCI [51-52]. Interventions in specific signaling pathways in B cells, astrocytes, and neuronal endosomal pain transmission may offer new therapeutics for SCI-pain. Endosomal pERK1/2-cyclin B1 signal transduction in the spinal neurons is a critical step in NMDAR pain transmission and the development of chronic pain [9, 23, 53, 54]. Early endosome antigen 1 (EEA1) and microtubule stabilization mediate their subcellular trafficking [33,54,55]. α-tubulin acetylation (a marker for hypermicrotubule stabilization) strongly enhances mitotic cell proliferation and EEA1 transport [28, 56]. Inhibition of α-tubulin acetylation and knockout of Atat1/MEC-17 (an inducer for α-tubulin acetylation [56]) were shown to reduce pain and intracellular endosomal trafficking [57]. Cyclin B1 not only mediates mitotic cell proliferation of B cells and astrocytes [35, 52], but also triggers postmitotic mitochondrial inhibition of ATP synthase and complex I oxidative damage, which is a key contributor to membrane hyper-excitability, causing neuropathic pain [23, 35, 58]. B cell autoimmune pain pathway has emerged and become an important activator to the neuronal endosomal pain transmission [45-49]. B cell proliferation and differentiate into plasma cells that produce antibody molecules (IgGs and IgMs). Autoantibodies target eeA1 and voltage-gated potassium channel (VGSK) in spinal cord and DRG, etc., causing pain directly by enhancing neuronal excitability and endosomal signal transport [48, 50]. B cell auto immune also induces astroglial proliferation [36, 38] that indirectly causes pain. Astroglial proliferation can release cytokines/chemokines (e.g., IL-1β, TNF-α, and IL-6, etc.) in the spinal cord to activate neuronal glutamate receptor pain transmission and enhance and prolong chronic SCI pain states [42, 59-61]. Most receptor antagonists activate the NMDAR and NK-1R at the surface of the neuronal membrane, but they are unable to effectively target intracellular endosomal NMDAR/NK-1R ongoing signal [53, 54]. ERK 1/₂ pathway inhibitors, such as PD98059 and U0126, cannot be used as drugs for reasons of toxicity, pharmacology, or solubility. B-cell depleting monoclonal antibodies have been developed for use in autoimmune diseases. However, these therapies are associated with severe toxicity [62, 63]. For targeting astroglial inflammation, there is increasing interest in the utilization of tubulin polymerization inhibitors such as colchicine [64]. Colchicine inhibits astroglial activation and pro-inflammatory cytokine release [35, 65]. However, colchicine has a narrow therapeutic window and exhibits significant toxicity [66, 67]. As a mild inhibitor of tubulin polymerization, FBZ (fenbendazole) and FluBZ are hypothesized to inhibit mitotic B cell and astrocyte proliferation and endosomal signal transport after SCI. FBZ and FluBZ are benzimidazole anthelmintics[19, 35]. Their primary mechanism is to bind tubulin, mildly inhibit microtubule formation and associated functions including mitosis and intracellular signal transport [19, 21, 35]. They bind to β-tubulin at a colchicine sensitive site that is distinct form that of Vinca alkaloids [21]. They do not induce depolymerization of existing microtubules and neuropathy which is a dosing-limiting toxicity of Vinca alkaloid. FBZ and FluBZ are highly safe for animals and humans based on their profiles of PKs [43, 69, 91]. FBZ and FluBZ are poorly soluble in aqueous systems and cause its very low bioavailability after oral treatment in rats, pigs, sheep, dogs or humans [70, 71, 91]. Treatment with oral or sc FluBZ-Hydroxypropyl-β-cyclodextrin (CD)-based solution to mice, rats and pigs significantly improved its systemic exposure [70, 71]. FBZ-CD formulation also increases solubility in vitro [90]. CD is commonly used reagent for enhancing bioavailability of lipophilic drugs and can be used in both liquid and solid dosage forms [72, 91]. The CD-based FluBZ or FBZ formulation has significant therapeutic implications in human use [70].

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1A-C shows FluBZ IP treatment reduced pain behaviors after excitotoxic SCI in rats. Behavior data showed that FluBZ IP treatment (left bar) for 7 days, starting at 3 hrs post-QUIS injury significantly delayed the onset of grooming behavior (FIG. 1A, **p<0.01), reduced grooming area of skin damage (FIG. 1B, ***p<0.001), and decreased grooming severity (FIG. 1C, **p<0.01), compared to vehicle treatment (right bar). For grooming onset and area data, data are presented as mean ±S.E.M. and analyzed with t-test. For grooming severity class data nonparametric data), data are presented as Median ±IQR. Group differences were evaluated by Mann-Whitney U test. ***p<0.001, **p<0.01. n=10 per group.

FIG. 2 shows FluBZ IP treatment prevented pain behaviors after excitotoxic SCI in rats. Behavior data showed that FluBZ IP treatment for 7 days, starting at 3 hrs post-excitotoxic injury, prevented the onset and incidence of painful grooming behavior, compared to vehicle treatment.

FIG. 3 shows FluBZ IP treatment reduced dorsal horn α-tubulin acetylation after excitotoxic SCI. Immunofluorescence analysis of spinal cord sections showed that excitotoxic injury increased dorsal horn acetylated α-tubulin signal intensity at 28 days after excitotoxic SCI (center bar, ###p<0.001), compared to sham group (right bar). FluBZ IP treatment (left bar) for 7 days, starting at 3 hrs post-excitotoxic injury, reduced the upregulation of α-tubulin acetylation (***p<0.001), compared to vehicle treatment (center bar) at the dorsal horn of spinal cord 4 weeks after excitotoxic injury in rats. Data are presented as mean ±S.E.M., n=4-5/group, and analyzed with ANOVA followed by Bonferroni post-hoc analysis.

FIG. 4 shows FluBZ IP treatment reduced dorsal horn α-tubulin acetyltransferase MEC-17 after excitotoxic SCI. Immunofluorescence analysis of spinal cord sections showed that excitotoxic injury increased dorsal horn α-tubulin acetyltransferase MEC-17 signal intensity at 28 days after excitotoxic SCI (center bar, ##p<0.01), compared to sham group (right bar). FluBZ IP treatment (left bar) for 7 days, starting at 3 hrs post-excitotoxic injury, reduced the upregulation of α-tubulin acetyltransferase MEC-17 (**p<0.01), compared to vehicle treatment (cener bar) at the dorsal horn of spinal cord 4 weeks after excitotoxic SCI in rats. Data are presented as mean ±S.E.M., n=4-5/group, and analyzed with ANOVA followed by Bonferroni post-hoc analysis.

FIG. 5 shows FluBZ IP treatment reduced dorsal horn endosomal EEA1-NMDAR1 dual signal intensity after excitotoxic SCI. Immunofluorescence analysis of spinal cord sections showed that excitotoxic injury increased dorsal horn endosomal EEA1-NMDAR1 dual signal intensity at 28 days after excitotoxic SCI (center bar, ###p<0.001), compared to sham group (right bar). FluBZ IP treatment (left bar) for 7 days, starting at 3 hrs post-excitotoxic injury, reduced the upregulation of endosomal EEA1-NMDAR1 dual signal intensity (***p<0.001), compared to vehicle treatment (center bar) at the dorsal horn of spinal cord 4 weeks after excitotoxic SCI in rats. Data are presented as mean ±S.E.M., n=4-5/group, and analyzed with ANOVA followed by Bonferroni post-hoc analysis.

FIG. 6 shows FluBZ IP treatment reduced dorsal horn endosomal EEA1-pERK1/2 dual signal intensity after excitotoxic SCI. Immunofluorescence analysis of spinal cord sections showed that excitotoxic injury increased dorsal horn endosomal EEA1-pERK1/2 dual signal intensity (merged) at 28 days after excitotoxic SCI (center bar, ###p<0.001), compared to sham group (right bar). FluBZ IP treatment (left bar) for 7 days, starting at 3 hrs post-excitotoxic injury, reduced the upregulation of endosomal EEA1-pERK1/2 dual signal intensity (***p<0.001), compared to vehicle treatment (center bar) at the dorsal horn of spinal cord 4 weeks after excitotoxic SCI in rats. Data are presented as mean ±S.E.M., n=4-5/group, and analyzed with ANOVA followed by Bonferroni post-hoc analysis

FIG. 7 shows FluBZ IP treatment reduced dorsal horn mitochondrial Cyclin b1 dual signal intensity after excitotoxic SCI. Immunofluorescence analysis of spinal cord sections showed that excitotoxic injury increased dorsal horn mitochondrial Cyclin b1 dual signal intensity at 28 days after excitotoxic SCI (center bar, ###p<0.001), compared to sham group (right bar). FluBZ IP treatment (left bar) for 7 days, starting at 3 hrs post-excitotoxic injury, reduced the upregulation of mitochondrial cyclin B1 dual signal intensity (***p<0.001), compared to vehicle treatment (center bar) at the dorsal horn of spinal cord 4 weeks after excitotoxic SCI in rats. Data are presented as mean ±S.E.M., n=4-5/group, and analyzed with ANOVA followed by Bonferroni post-hoc analysis.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “treatment” refers to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As will be understood by those of ordinary skill in the art, when the term “prevent” or “prevention” is used in connection with a prophylactic treatment, it should not be understood as an absolute term that would preclude any modicum of pain in a subject. Rather, as used in the context of prophylactic treatment, the term “prevent” can refer to inhibiting the development of or limiting the severity of, arresting the development of pain, and the like.

As used herein, the term “diagnosed” means having been subjected to a physical examination by a person of skill, for example, a physician, and found to have a condition that can be diagnosed or treated by the compounds, compositions, or methods disclosed herein. Such a diagnosis can be in reference to a disorder, such as neuropathic pain, and the like, as discussed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular subject will depend upon a variety of factors including the disorder being treated and the severity of the disorder; the specific composition employed; the age, body weight, general health, sex and diet of the subject; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

One embodiment of the present invention is a method for treating neuropathic pain in a patient with a traumatic spinal cord injury comprising administering an effective amount of flubendazole to a patient suffering from a traumatic spinal cord injury.

A further embodiment of the present invention is a method of treating neuropathic pain in a patient with a traumatic spinal cord injury comprising administering an effective amount of a benzimidazoles, such as, benzimidazole, albendazole, thiabendazole, ciclobenzazole, or fenbendazole to a patient suffering from a traumatic spinal cord injury.

A further embodiment of the present invention is a method of treating neuropathic pain in a patient with a traumatic spinal cord injury comprising administering an effective amount of an α-tubulin acetylation inhibitor to a patient suffering from a traumatic spinal cord injury.

Another embodiment of the present invention is a method of treating neuropathic pain in a patient with a traumatic spinal cord injury comprising administering an effective amount of an α-tubulin acetylation inhibitor to a patient suffering from a traumatic spinal cord injury.

A further embodiment of the present invention is a method of treating neuropathic pain in a patient with a traumatic spinal cord injury comprising administering an effective amount of an endosomal NR1 and pERK1/2 inhibitor to a patient suffering from a traumatic spinal cord injury.

Another embodiment of the present invention is a method of treating neuropathic pain in a patient with a traumatic spinal cord injury comprising administering an effective amount of a mitochondrial cyclin b1 inhibitor to a patient suffering from a traumatic spinal cord injury.

A further embodiment of the present invention is a method of treating neuropathic pain in a patient with a spinal cord injury comprising administering an effective amount of a microtubule destabilizing pharmaceutical agent to a patient suffering from a traumatic spinal cord injury.

Another embodiment of the present invention includes a method of preventing neuropathic pain in a patient with a spinal cord injury at risk for developing neuropathic pain comprising administrating an effective amount of flubendazole to a patient with a spinal cord injury at risk of developing neuropathic pain.

### EXAMPLES

### Materials & Methods

### Animals

Male Sprague-Dawley (SD) adult rats at 3 months, weighing 220-250 g, were used (Charles River, Indianapolis, Ind.). Rats were kept under standard housing conditions (12:12 light and dark cycle) with food and water ad libitum for at least 1 week following arrival in an enclosed, pathogen-free animal facility. All experimental procedures were approved and carried out in accordance with the Guidelines of the US National Institutes of Health and Institutional Animal Care and Use Committee (IACUC) of the University of Kentucky.

### Chemicals

Flubendazole (FluBZ), quisqualic acid (QUIS), fish gelatin, and anti-acetylated α-Tubulin antibody (T7451, mouse monoclonal) were purchased from Sigma-Aldrich (St. Louis, Mo.). Anti-phospho-p44/42 MAPK (pERK1/2) antibody (#9101s, rabbit polyclonal) and anti-cyclin B1 antibody (#4138, rabbit polyclonal) were purchased from Cell Signaling Technology, Inc. (Danvers, Mass.). Anti-acetyltransferase ATAT1/MEC-17 antibody (ab58742, rabbit polyclonal), anti-NMDAR1 antibody (ab17345, rabbit polyclonal), and anti-VDAC1 antibody (ab14734, mouse monoclonal) were purchased from Abcam (Cambridge, Mass.). Anti-EEA1 antibody (14-9114-82, mouse monoclonal), Donkey anti-rabbit Alexa Fluor 594 antibodies, goat anti-mouse Alexa Fluor 488 antibodies and Superfrost/Plus microscopy slides were purchased from Thermo Scientific (Waltham, Mass.). Hoechst and vectashield mounting media were purchased from Vector Lab (CA, USA).

### Excitotoxic SCI

Excitotoxic SCI was produced by intraspinal injection of the glutamatergic AMPA/metabotropic receptor agonist quisqualic acid (QUIS) as previously described [10, 11]. Briefly, rats were anesthetized with a mixture of ketamine and xylazine (intraperitoneally, IP). Animals were placed in a stereotaxic unit and the spinal column immobilized with a vertebral clamp. One injection window for intraspinal injection was made by laminectomy between spinal segments T12-L1. Injections of 125 mM QUIS was made unilaterally at three levels of the cord by convection enhanced delivery. Injections, spaced 0.3 mm apart, were made at a depth of 1000 μm below the surface in segments T12-L1 between the dorsal root entry zone and the dorsal vein. These coordinates place injections in the center of the gray matter between spinal laminae IV-VI. The total volume of QUIS injected was 1.2 μl (0.4 μl each site, delivered over 4 minutes). Following injections, muscles were sutured over the laminectomy site, and the skin closed with wound clips.

### Groups and Drug Administration

Twenty-four male SD rats were randomly assigned to three groups: (A) FluBZ treatment plus intraspinal injection of QUIS (n=10); (B) vehicle treatment plus intraspinal injection of QUIS (n=10); and (C) Sham operation (intraspinal injection of saline, n=4). Assessment of excessive grooming behavior was used to evaluate the effects of FluBZ or vehicle on the development of the spontaneous pain behavior following excitotoxic SCI. The survival time of animals consisted of a period of 4 weeks or until tissue damage progressed beyond the superficial layers of skin, at which time (<4 weeks) animals were euthanized. FluBZ (10 mg/kg/day) or vehicle was intraperitoneally injected once daily for a period of 7 days post-injection of QUIS. The FluBZ or vehicle treatment began at 3 h post-QUIS injection. FluBZ (Sigma) was dissolved in 0.9% saline plus 0.01% Tween 80 (Vehicle).

Assessment of spontaneous pain-related excessive grooming behavior

Assessment of the spontaneous pain-related excessive grooming behavior was performed as described in previous studies [11, 18]. Beginning on the second day after QUIS injection, animals were examined daily for signs of excessive grooming in dermatomes associated with the injected spinal segment, i.e. self-directed biting and scratching resulting in damage to the superficial layers of skin. Once hair loss and early signs of skin damage were observed, the area was reconstructed on a standardized drawing. Assessment of grooming behavior continued for a period of 4 weeks or until tissue damage progressed beyond the superficial layers of skin (Class IV), at which time animals were euthanized. The severity of overgrooming pain behavior was divided into 4 classes: Class I, hair removal over contiguous portions of a dermatome; Class II, extensive hair removal combined with signs of damage to superficial layers of skin; Class III, hair removal and damage to dermal layers of skin; Class IV, subcutaneous tissue damage (experiment terminated). Quantitative analysis of spontaneous excessive grooming pain behavior consisted of the incidence, onset time, mean area of skin damage, and severity of overgrooming pain behaviors for all animals as previously described [18].

### Spinal Cord Tissue Process

At 4 weeks post-QUIS injury, five FluBZ or vehicle-treated rats and four sham rats were euthanized with an overdose of pentobarbital and transcardially perfused with ice cold phosphate-buffered saline (PBS) followed by phosphate-buffered 4% paraformaldehyde. Spinal cords were removed and post-fixed in the same fixative overnight, then placed in 30% sucrose-PBS solution for cryoprotection. The fixed spinal cords (1.5 cm in length) centered at the QUIS injection site were serially cryosectioned at a thickness of 20 μm. Every fifth section (interval between 100 μm) was mounted onto each gelatin-coated slide. The interval between two sections on each slide is 1 mm. 10 sets of slides were collected and stored at −20° C.

### Immunofluorescence Staining

Immunofluorescence staining was performed as previously described [19, 20]. Briefly, spinal cord sections at QUIS lesion site between T12 and L1 (2 mm rostral and caudal to the lesion epicenter) were incubated overnight in TBS with 0.1% Triton-X-100, 5% normal goat serum, 5% normal donkey serum, and 0.1% fish gelatin containing one of the primary rabbit polyclonal antibodies (ATAT1/MEC-17, NMDAR1, pERK1/2, and cyclin B1, 1:250) and mouse monoclonal antibodies (acetylated α-tubulin, EEA1, and VDAC1, 1:250). The spinal section samples were washed 3×10 min in TBS with 0.1% Triton-X-100 and followed by incubation with donkey anti-rabbit Alexa Fluor 594 conjugated secondary antibody (1:1000) and goat anti-mouse Alexa Fluor 488 conjugated secondary antibody (1:1000) for 1 h and then Hoechst (1:1000) for 20 min in TBS at room temperature. After washing three times in TBS, all fluorescently labeled sections were mounted on Superfrost/Plus microscopy slides, air-dried and coverslipped using Vectashield mounting media.

### Confocal Microscopy and Image Capturing

All fluorescent sections were visualized and imaged using a laser scanning confocal microscopy system (Nikon C2+, Melville, N.Y., USA). Images were acquired at the laser setting: HV 100 and 3.0 percent laser for acetylated α-tubulin, HV 115 and 3.00 percent laser for acetyltransferase MEC-17, HV 115 and 3.0 percent laser for EEA1, HV 115 and 3.0 percent laser for pERK1/2, HV 120 and 3.0 percent laser for cyclin B1, HV 115 and 3.0 percent laser for VDAC1, HV 115 and 3.0 percent laser for NMDAR1. The fluorescent signals within the spinal cord section (1024×1024 μm) in the dorsal horn of spinal cord (region of interest, ROI) were captured (magnification 10×). Double or triple-labeled sections for each target signal was visualized and imaged within Z-stacks of 6 images with ˜3 μm steps corresponding to a 20-μm depth for each ROI.

### Stereological Analysis

The stereological analysis of image data within dorsal horn of spinal cord in sham or QUIS-injured animals with treatment of FluBZ or vehicle will be performed using NIS-Elements AR program. Serial double-labeled immunofluorescent signal images of spinal cord sections at lesion epicenter and 2 mm rostral and caudal to the lesion site were analyzed using methods of Binary Layers and Automated Measurement Results. Using a threshold setting of 1251 for acetylated α-tubulin and acetyltransferase MEC-17, 650 for EEA1, 406 for NMDAR1, 1999 for cyclin B1, and 1706 for VDAC1, Number of Object and Signal Intensity were measured. Stereological estimation for Number of Object and Mean Intensity of dual-labeled signal in the dorsal horn of spinal cord section were performed within Z-stacks of 6 images with ˜3.3-μm steps corresponding to a 20-μm depth for each ROI in a blinded manner. Four independent sections were evaluated for each ROI per animal (four sections separated 100 um apart per animal). For each animal, the calculated fluorescent number of object or mean intensity for expression of each target of four sections was averaged. Subsequently, the average number of object or intensity per section of all animals in each group was averaged to obtain a mean intensity value. Results are presented at Mean±SEM.

### Statistical Analysis

Data related to the onset time, area of grooming-induced skin damage, and immunofluorescent number of object and mean intensity are expressed as mean ±standard error of mean (SEM) and analyzed by using a commercially available computer program (StatView; SAS Institute, Inc, Cary, N.C.). Group differences were evaluated by repeated measures ANOVA followed by the Bonferroni post hoc test or t-test. In the analysis of grooming severity data (class I to IV), values are expressed as median±IQR (interquartile range). For the analysis of these data the nonparametric Kruskal-Wallis test was used for multiple comparisons followed by the Mann-Whitney U test to compare individual groups. All behavioral and molecular testing and analyses were carried out by individuals blinded to the drug treatment of each animal. P values of less than 0.05 were used for statistical significance.

### Results

### Microtubule Destabilization by FluBZ Prevented and Attenuated Spontaneous Pain After Excitotoxic SCI

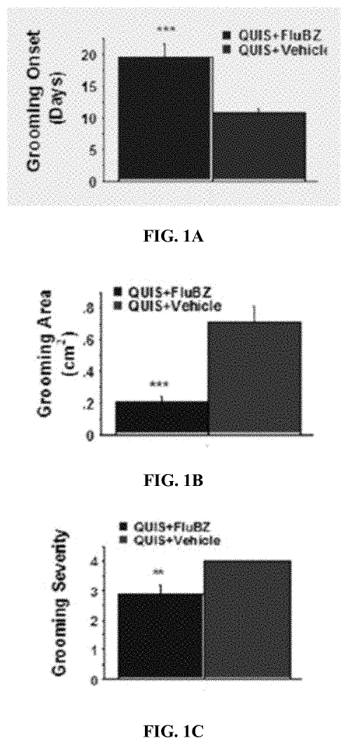

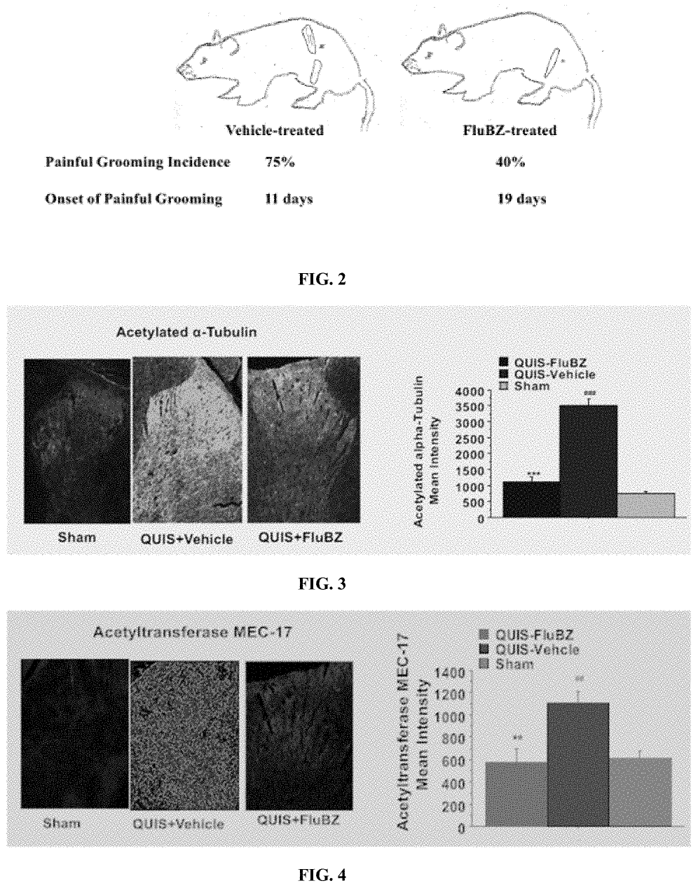

To examine whether microtubule destabilizing strategy has any anti-nociceptive role after excitotoxic spinal injury, we intraperitoneally (IP) administered microtubule destabilizing drug FluBZ and evaluated spontaneous pain behaviors after excitotoxic SCI. FluBZ or vehicle treatment began at 3 h post-excitotoxic injury, once daily for 7 days. Excitotoxic SCI-injured animals receiving vehicle treatment developed excessive grooming pain behaviors with an average onset of 11 days after excitotoxic injury (FIG. 1A and FIG. 2). FluBZ treatment significantly delayed the onset of excessive grooming pain behaviors (19 days after excitotoxic injury, FIG. 1A and FIG. 2) and reduced the incidence of pain behavior in excitotoxic SCI-injured rats from an incidence of 75% in vehicle-treated control rats to an incidence of 40% in FluBZ-treated rats (FIG. 2). The skin damage area and severity targeted for excessive grooming pain behaviors in vehicle-treated QUIS-injured animals showed a significant progression over time (FIG. 2). Excessive grooming pain behaviors in these vehicle-treated excitotoxic-injured animals occurred primarily ipsilateral to the spinal injury, began with biting and scratching of the skin dermatomes associated with spinal segments at or caudal to the site of injury, continued with removal of hair, and progressed to include damage to the dermal layers or subcutaneous tissue (Class IV) (FIG. 1C and FIG. 2). FluBZ administration (10 mg/kg/day) for one week post-excitotoxic injury resulted in a significant reduction in size of the grooming area (FIG. 1B and severity of grooming class (FIG. 1C). The results suggest that FluBZ IP administration prevents and attenuates chronic neuropathic pain after excitotoxic SCI in rats.

Microtubule destabilization by FluBZ reduced α-tubulin acetylation and α-tubulin acetyltransferase MEC-17 activity in spinal dorsal horn after excitotoxic SCI

To investigate whether FluBZ modulates microtubule stabilization, we examined acetylation of α-tubulin (a major marker of microtubule hyper-stabilization) and expression of α-tubulin N-acetyltransferase ATAT1/MEC-17 in the spinal dorsal horn following excitotoxic SCI by immunofluorescence staining analysis. α-tubulin N-acetyltransferase ATAT1 (also called α-tubulin K40 acetylase MEC-17, mechanosensory abnormality 17) is a specific inducer of α-tubulin acetylation. As shown in FIGS. 3 and 4, Immunofluorescence staining with confocal microscopy image analysis revealed that excitotoxic injury up-regulated robust α-tubulin acetylation (###p<0.001) and α-tubulin N-acetyltransferase MEC-17 (##p<0.01) in the spinal dorsal horn at lesion epicenter and 2 mm rostral and caudal to the lesion epicenter on day 28 following excitotoxic SCI, compared to sham controls. The levels of fluorescent signals in Number of Object, Object Area, ROI Area, Area Fraction, and Mean Intensity of α-tubulin acetylation and α-tubulin N-acetyltransferase MEC-17 were significantly elevated in spinal cord dorsal horn, especially in the superficial laminae (I-III) and IV-V laminae 4 weeks post-QUIS injury using a specific antibody against acetylated α-tubulin (FIG. 3) and α-tubulin N-acetyltransferase MEC-17 (FIG. 4). Many more acetylated α-tubulin-immunoreactive cells and MEC-17-immunoreactive cells were found in the ipsilateral spinal cord after excitotoxic injury.

Although most-positive cells were found in the superficial (Laminae I-III) dorsal horn, many positive cells were also seen in the laminae IV-V of the dorsal horn. Sham operation did not produce significant changes of α-tubulin acetylation or acetyltransferase MEC-17 in the spinal dorsal horn. Although their upregulation was predominantly found in the ipsilateral spinal cord, a moderate increase in the contralateral superficial dorsal horn was also evident. Post-treatment with FluBZ (10 m/kg/day for 7 days) 3 h post-QUIS injury significantly decreased expression of acetyltransferase MEC-17 (**p<0.01) and α-tubulin acetylation (***p<0.001) in the spinal dorsal horn of spinal cord at lesion site 28 days following QUIS injury. The results suggest that FluBZ reduces α-tubulin acetylation and α-tubulin acetyltransferase MEC-17 activity in the spinal dorsal horn following excitotoxic SCI.

### Microtubule Destabilization by FluBZ Reduced Endosomal NR1 Signal in Spinal Dorsal Horn After Excitotoxic SCI

Our previous study suggests that NMDA receptor subunit NR1 expression is upregulated in spinal cord following excitotoxic injury in rats [10]. To investigate whether the NR1 continues to signal in endosomes after excitotoxic SCI, co-localization of NR1 with early endosomal marker EEA1 was examined following excitotoxic injury. Immunofluorescent confocal imaging data showed that both individual-labeled signal levels and double-labeled signal levels (Number Object, Binary Area, Binary Area Fraction, ROI Area, and Mean Intensity) of NR1 and/or EEA1 in spinal dorsal horn were significantly elevated (FIG. 5, ###p<0.001) 28 days following excitotoxic injury, compared to sham control. Furthermore, Microtubule destabilization drug FluBZ was used to investigate whether microtubule destabilization reduces endosomal NR1 signaling transduction in rat model of excitotoxic SCI. Immunofluorescent confocal imaging data showed that excitotoxic injury-induced endosomal EEA1-NR1 fluorescent signals in the dorsal horn at 28 days post-excitotoxic injury were significantly suppressed by post-treatment of the microtubule destabilizing drug FluBZ FIG. 5, ***p<0.001). This result suggests that microtubule destabilization by FluBZ reduces endosomal NR1 signal in the dorsal horn following excitotoxic injury.

Microtubule Destabilization by FluBZ Reduces Endosomal pERK1/2 Signal in Spinal Dorsal Horn After Excitotoxic SCI

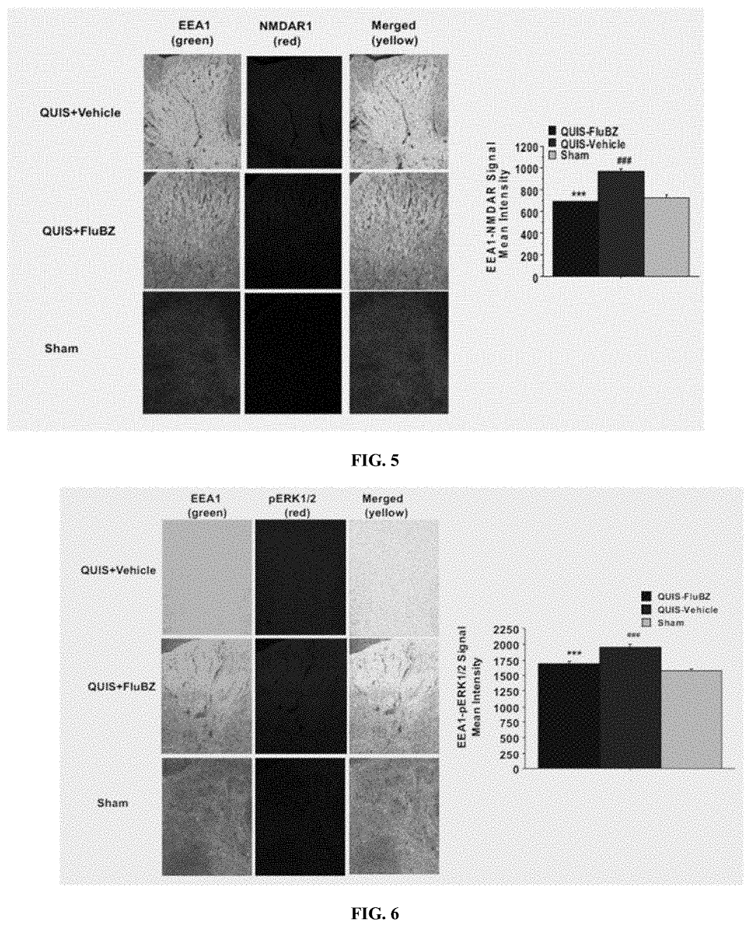

Endosomal ERK1/2 activation in dorsal horn of spinal cord is a key target of NMDAR and NK-1R that is a central process in the subcellular pain signaling transport [9, 10, 21]. To investigate whether microtubule destabilization inhibits endosomal ERK1/2 activation in the spinal dorsal horn, ERK1/2 phosphorylation and co-localization with EEA1 were examined at 28 days following excitotoxic injury in the presence or absence of FluBZ treatment. Phospho-ERK1/2 immunofluorescent signal, EEA1 immunofluorescent signal and dual-labeled phospho-ERK1/2 and EEA1 immunofluorescent signals were significantly increased in spinal cord dorsal horn at 28 days following excitotoxic injury (FIG. 6, ###p<0.001), compared to sham controls. The immunofluorescence data suggested that excitotoxic injury resulted in significant and sustained dorsal horn endosomal ERK1/2 activation. FluBZ IP treatment significantly inhibited excitotoxic injury-induced endosomal EEA1-pERK1/2 fluorescent binary number object, binary area fraction, binary area, and mean intensity in the dorsal horn at 28 days post-QUIN injury (FIG. 6, ***p<0.001). The data suggested that microtubule destabilization by FluBZ reduced endosomal pERK1/2 signal in spinal dorsal horn after excitotoxic SCI.

### Microtubule Destabilization by FluBZ Reduced Mitochondrial Cyclin B1 Activity in Spinal Dorsal Horn After Excitotoxic SCI

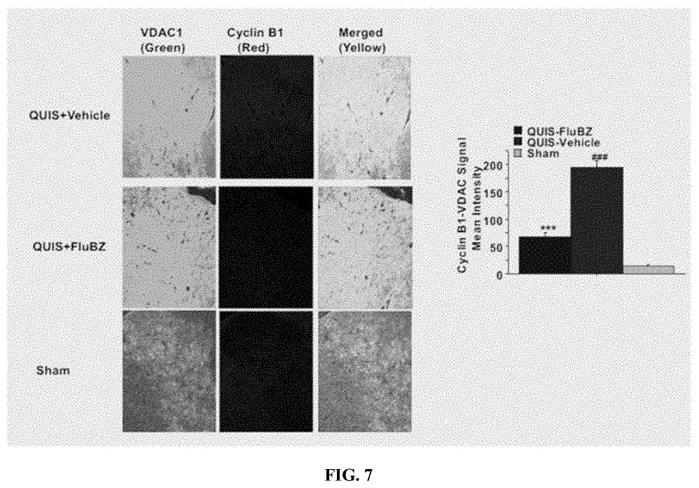

Excitotoxic damage-induced mitochondrial Cyclin B1 accumulation has been shown to contribute to mitochondrial dysfunction [24, 25]. In the present study, we examined the levels of mitochondrial cyclin B1 in the spinal dorsal horn following excitotoxic injury using antibodies specifically against cyclin B1 or VDAC1 (a marker for mitochondria) in order to determine whether mitochondrial Cyclin B1 is involved in pain signaling cascades after excitotoxic injury. The activity of cyclin B1 and co-localization with VDAC1 in the spinal dorsal horn were evaluated by double immunofluorescence staining and confocal microscopy imaging analysis using antibodies that selectively recognizes Cyclin B1 or VDAC1 protein on the dorsal horn. Results showed that dual-labeled fluorescence levels of number object, binary area fraction, binary area, ROI area, and mean intensity of Cyclin b 1 and VDAC1 increased 28 days after excitotoxic injury (FIG. 7, ###p<0.001), compared to sham-operated animals. FluBZ post-injury administration (10 mg/kg/day for 7 days) significantly reduced mitochondrial cyclin b1 activity in the dorsal horn 28 days following excitotoxic injury (FIG. 7, ***p<0.001), compared to vehicle-treated animals. The results suggested that microtubule destabilization by FluBZ reduced mitochondrial cyclin B1 activity in spinal dorsal horn after excitotoxic SCI.

### REFERENCES

All publications, patents, and patent applications mentioned in this specification are herein incorporated by reference to the same extent as if each individual publication, patent, or patent application was specifically and individually indicated to be incorporated by reference, including the references set forth in the following list:
1. Morley, S. J., et al., Acetylated tubulin is essential for touch sensation in mice. Elife, 2016. 5.
2. Li, Y., et al., The Cancer Chemotherapeutic Paclitaxel Increases Human and Rodent Sensory Neuron Responses to TRPV1 by Activation of TLR4. J Neurosci, 2015. 35(39): p. 13487-500.
3. Bryce, T. N., et al., International spinal cord injury pain classification: part I. Background and description. March 6-7, 2009. Spinal Cord, 2012. 50(6): p. 413-7.
4. Schomberg, D., et al., Spinal cord injury induced neuropathic pain: Molecular targets and therapeutic approaches. Metab Brain Dis, 2015.
5. Yezierski, R. P., Pain following spinal cord injury: the clinical problem and experimental studies. Pain, 1996. 68(2-3): p. 185-94.
6. Teasell, R. W., et al., A systematic review of pharmacologic treatments of pain after spinal cord injury. Arch Phys Med Rehabil, 2010. 91(5): p. 816-31.
7. Hatch, M. N., et al., Neuropathic pain and SCI: Identification and treatment strategies in the 21st century. J Neurol Sci, 2018. 384: p. 75-83.
8. Gao, Y. J. and R. R. Ji, c-Fos and pERK, which is a better marker for neuronal activation and central sensitization after noxious stimulation and tissue injury? Open Pain J, 2009. 2: p. 11-17.
9. Ji, R. R., et al., MAP kinase and pain. Brain Res Rev, 2009. 60(1): p. 135-48.
10. Yu, C. G. and R. P. Yezierski, Activation of the ERK1/2 signaling cascade by excitotoxic spinal cord injury. Brain Res Mol Brain Res, 2005. 138(2): p. 244-55.
11. Yezierski, R. P., et al., Excitotoxic spinal cord injury: behavioral and morphological characteristics of a central pain model. Pain, 1998. 75(1): p. 141-55.
12. Lee, J. W., et al., Predifferentiated GABAergic neural precursor transplants for alleviation of dysesthetic central pain following excitotoxic spinal cord injury. Front Physiol, 2012. 3: p. 167.
13. Bansal, Y. and O. Silakari, The therapeutic journey of benzimidazoles: a review. Bioorg Med Chem, 2012. 20(21): p. 6208-36.
13. Byrnes, K. R., D. J. Loane, and A. I. Faden, Metabotropic glutamate receptors as targets for multipotential treatment of neurological disorders. Neurotherapeutics, 2009. 6(1): p. 94-107.
14. Al-Bassam, J. and K. D. Corbett, alpha-Tubulin acetylation from the inside out. Proc Natl Acad Sci U S A, 2012. 109(48): p. 19515-6.
15. Li, Y., et al., MAPK signaling downstream to TLR4 contributes to paclitaxel-induced peripheral neuropathy. Brain Behav Immun, 2015. 49: p. 255-66.
16. Bhuwania, R., A. Castro-Castro, and S. Linder, Microtubule acetylation regulates dynamics of KIF1C-powered vesicles and contact of microtubule plus ends with podosomes. Eur J Cell Biol, 2014. 93(10-12): p. 424-37.
17. Yu, C. G., et al., Effects of agmatine, interleukin-10, and cyclosporin on spontaneous pain behavior after excitotoxic spinal cord injury in rats. J Pain, 2003. 4(3): p. 129-40.
18. Yu, C. G., et al., Involvement of ERK2 in traumatic spinal cord injury. J Neurochem, 2010. 113(1): p. 131-42.
19. Yu, C. G., et al., Fenbendazole improves pathological and functional recovery following traumatic spinal cord injury. Neuroscience, 2014. 256: p. 163-9.
20. Jensen, D. D., et al., Neurokinin 1 receptor signaling in endosomes mediates sustained nociception and is a viable therapeutic target for prolonged pain relief. Sci Transl Med, 2017. 9(392).
21. Spagnuolo, P. A., et al., The antihelmintic flubendazole inhibits microtubule function through a mechanism distinct from Vinca alkaloids and displays preclinical activity in leukemia and myeloma. Blood, 2010. 115(23): p. 4824-33.
22. Patel, S. P., et al., Differential effects of the mitochondrial uncoupling agent, 2,4-dinitrophenol, or the nitroxide antioxidant, Tempol, on synaptic or nonsynaptic mitochondria after spinal cord injury. J Neurosci Res, 2009. 87(1): p. 130-140.
23. Veas-Perez de Tudela, M., et al., Regulation of Bc1-xL-ATP Synthase Interaction by Mitochondrial Cyclin B1-Cyclin-Dependent Kinase-1 Determines Neuronal Survival. J Neurosci, 2015. 35(25): p. 9287-301.
24. Maestre, C., et al., Cdk5 phosphorylates Cdh1 and modulates cyclin B1 stability in excitotoxicity. EMBO J, 2008. 27(20): p. 2736-45.
25. Flatters, S. J., The contribution of mitochondria to sensory processing and pain. Prog Mol Biol Transl Sci, 2015. 131: p. 119-46.
26. Patel, S. P., et al., Acetyl-L-carnitine ameliorates mitochondrial dysfunction following contusion spinal cord injury. J Neurochem, 2010. 114(1): p. 291-301.
27. Gupta, R. S. and A. K. Dudani, Mechanism of action of antimitotic drugs: a new hypothesis based on the role of cellular calcium. Med Hypotheses, 1989. 28(1): p. 57-69.
28. Hoepfner, S., et al., Modulation of receptor recycling and degradation by the endosomal kinesin KIF16B. Cell, 2005. 121(3): p. 437-50.
29. Cane, M., et al., Tubulin is an inherent component of mitochondrial membranes that interacts with the voltage-dependent anion channel. J Biol Chem, 2002. 277(37): p. 33664-9.
30. Misawa, T., et al., Microtubule-driven spatial arrangement of mitochondria promotes activation of the NLRP3 inflammasome. Nat Immunol, 2013. 14(5): p. 454-60.
31. Zhou, R., et al., A role for mitochondria in NLRP3 inflammasome activation. Nature, 2011. 469(7329): p. 221-5.
32. Misawa, T., et al., Resveratrol inhibits the acetylated alpha-tubulin-mediated assembly of the NLRP3-inflammasome. Int Immunol, 2015. 27(9): p. 425-34.
33. Wolff, J., Plasma membrane tubulin. Biochim Biophys Acta, 2009. 1788(7): p. 1415-33.
34. Wu, C., et al., A functional dynein-microtubule network is required for NGF signaling through the Rap1/MAPK pathway. Traffic, 2007. 8(11): p. 1503-20.
35. Yu, C. G., et al., Repositioning Flubendazole for Spinal Cord Injury. J Neurotrauma, 2019.
36. Ankeny, D. P., Z. Guan, and P. G. Popovich, B cells produce pathogenic antibodies and impair recovery after spinal cord injury in mice. J Clin Invest, 2009. 119(10): p. 2990-9.
37. Ankeny, D. P. and P. G. Popovich, B cells and autoantibodies: complex roles in CNS injury. Trends Immunol, 2010. 31(9): p. 332-8.
38. Ulndreaj, A., et al., Characterization of the Antibody Response after Cervical Spinal Cord Injury. J Neurotrauma, 2017. 34(6): p. 1209-1226.
39. Casili, G., et al., B-Cell Depletion with CD20 Antibodies as New Approach in the Treatment of Inflammatory and Immunological Events Associated with Spinal Cord Injury. Neurotherapeutics, 2016. 13(4): p. 880-894.
40. Dekaban, G. A. and S. Thawer, Pathogenic antibodies are active participants in spinal cord injury. J Clin Invest, 2009. 119(10): p. 2881-4.
41. Walters, E. T., Neuroinflammatory contributions to pain after SCI: roles for central glial mechanisms and nociceptor-mediated host defense. Exp Neurol, 2014. 258: p. 48-61.
42. Gao, Y. J. and R. R. Ji, Targeting astrocyte signaling for chronic pain. Neurotherapeutics, 2010. 7(4): p. 482-93.
43. Karimi-Abdolrezaee, S. and R. Billakanti, Reactive astrogliosis after spinal cord injury beneficial and detrimental effects. Mol Neurobiol, 2012. 46(2): p. 251-64.
44. Ji, R. R., T. Berta, and M. Nedergaard, Glia and pain: Is chronic pain a gliopathy? Pain, 2013.
45. Xu, M., et al., Pain and the immune system: emerging concepts of IgG-mediated autoimmune pain and immunotherapies. J Neurol Neurosurg Psychiatry, 2018.
46. Dawes, J. M. and A. Vincent, Autoantibodies and pain. Curr Opin Support Palliat Care, 2016. 10(2): p. 137-42.
47. David Clark, J., et al., Autoinflammatory and autoimmune contributions to complex regional pain syndrome. Mol Pain, 2018. 14: p. 1744806918799127.
48. Klein, C. J., et al., Chronic pain as a manifestation of potassium channel-complex autoimmunity. Neurology, 2012. 79(11): p. 1136-44.
49. Bennett, D. L. and A. Vincent, Autoimmune pain: an emerging concept. Neurology, 2012. 79(11): p. 1080-1.
50. Selak, S., et al., Human autoantibodies against early endosome antigen-1 enhance excitatory synaptic transmission. Neuroscience, 2006. 143(4): p. 953-64.
51. Basbaum, A. I., et al., Cellular and molecular mechanisms of pain. Cell, 2009. 139(2): p.26′7-84.
52. Wu, J., et al., Cell cycle inhibition limits development and maintenance of neuropathic pain following spinal cord injury. Pain, 2016. 157(2): p. 488-503.
53. Jensen, D. D., et al., Neurokinin 1 receptor signaling in endosomes mediates sustained nociception and is a viable therapeutic target for prolonged pain relief. Sci Transl Med, 2017. 9(392).
54. Yarwood, R. E., et al., Endosomal signaling of the receptor for calcitonin gene-related peptide mediates pain transmission. Proc Natl Acad Sci U S A, 2017. 114(46): p. 12309-12314.
55. Washbourne, P., et al., Cycling of NMDA receptors during trafficking in neurons before synapse formation. J Neurosci, 2004. 24(38): p. 8253-64.
56. Bhuwania, R., A. Castro-Castro, and S. Linder, Microtubule acetylation regulates dynamics of KIF1C-powered vesicles and contact of microtubule plus ends with podosomes. Eur J Cell Biol, 2014. 93(10-12): p. 424-37.
57. Morley, S. J., et al., Acetylated tubulin is essential for touch sensation in mice. Elife, 2016.5.
58. Khangura, R. K., et al., An integrated review on new targets in the treatment of neuropathic pain. Korean J Physiol Pharmacol, 2019. 23(1): p. 1-20.
59. Walters, E. T., Neuroinflammatory contributions to pain after SCI: Roles for central glial mechanisms and nociceptor-mediated host defense. Exp Neurol, 2014. 258C: p. 48-61.
60. Nakagawa, T. and S. Kaneko, Spinal astrocytes as therapeutic targets for pathological pain. J Pharmacol Sci, 2010. 114(4): p. 347-53.
61. Clark, A. K., E. A. Old, and M. Malcangio, Neuropathic pain and cytokines: current perspectives. J Pain Res, 2013. 6: p. 803-14.
62. Dorner, T., N. Kinnman, and P. P. Tak, Targeting B cells in immune-mediated inflammatory disease: a comprehensive review of mechanisms of action and identification of biomarkers. Pharmacol Ther, 2010. 125(3): p. 464-75.
63. Levesque, M. C. and E. W. St Clair, B cell-directed therapies for autoimmune disease and correlates of disease response and relapse. J Allergy Clin Immunol, 2008. 121(1): p. 13-21; quiz 22-3.
64. Leung, Y. Y., L. L. Yao Hui, and V. B. Kraus, Colchicine--Update on mechanisms of action and therapeutic uses. Semin Arthritis Rheum, 2015. 45(3): p. 341-50.
65. Cocco, G., D. C. Chu, and S. Pandolfi, Colchicine in clinical medicine. A guide for internists. Eur J Intern Med, 2010. 21(6): p. 503-8.
66. Jayaprakash, V., G. Ansell, and D. Galler, Colchicine overdose: the devil is in the detail. N Z Med J, 2007. 120(1248): p. U2402.
67. Zughaier, S., et al., Potent anti-inflammatory activity of novel microtubule-modulating brominated noscapine analogs. PLoS One, 2010. 5(2): p. e9165.
68. Barron, S., et al., The behavioral teratogenic potential of fenbendazole: a medication for pinworm infestation. Neurotoxicol Teratol, 2000. 22(6): p. 871-7.
69. Villar, D., et al., Biologic effects of fenbendazole in rats and mice: a review. J Am Assoc Lab Anim Sci, 2007. 46(6): p. 8-15.
70. Ceballos, L., et al., Exploring the potential of flubendazole in filariasis control: evaluation of the systemic exposure for different pharmaceutical preparations. PLoS Negl Trop Dis, 2014. 8(5): p. e2838.
71. Ceballos, L., et al., Pharmacokinetic comparison of different flubendazole formulations in pigs: A further contribution to its development as a macrofilaricide molecule. Int J Parasitol Drugs Drug Resist, 2015. 5(3): p. 178-84.
72. Wu, Y., et al., Delayed post-injury administration of riluzole is neuroprotective in a preclinical rodent model of cervical spinal cord injury. J Neurotrauma, 2013. 30(6): p. 441-52.
73. Bareiss, S. K., E. Dugan, and K. L. Brewer, PI3K mediated activation of GSK-3beta reduces at-level primary afferent growth responses associated with excitotoxic spinal cord injury dysesthesias. Mol Pain, 2015. 11: p. 35.
74. Bareiss, S. K., et al., Excitotoxic spinal cord injury induced dysesthesias are associated with enhanced intrinsic growth of sensory neurons. Neurosci Lett, 2013. 542: p. 113-7.
75. Landis, S. C., et al., A call for transparent reporting to optimize the predictive value of preclinical research. Nature, 2012. 490(7419): p. 187-91.
76. Collins, F. S. and L. A. Tabak, Policy: NIH plans to enhance reproducibility. Nature, 2014. 505(7485): p. 612-3.
77. Muhlhausler, B. S., F. H. Bloomfield, and M. W. Gillman, Whole animal experiments should be more like human randomized controlled trials. PLoS Biol, 2013. 11(2): p.e1001481.
78. Kilkenny, C. and D. G. Altman, Improving bioscience research reporting: ARRIVE-ing at a solution. Lab Anim, 2010. 44(4): p. 377-8.
79. Lemmon, V. P., et al., Minimum Information about a Spinal Cord Injury Experiment: A Proposed Reporting Standard for Spinal Cord Injury Experiments. J Neurotrauma, 2014.
80. Yu, C. G., et al., Calpain 1 knockdown improves tissue sparing and functional outcomes after spinal cord injury in rats. J Neurotrauma, 2013. 30(6): p. 427-33.
81. Kwon, B. K., et al., A grading system to evaluate objectively the strength of pre-clinical data of acute neuroprotective therapies for clinical translation in spinal cord injury. JNeurotrauma, 2011. 28(8): p. 1525-43.
82. Metz, G. A., et al., Validation of the weight-drop contusion model in rats: a comparative study of human spinal cord injury. J Neurotrauma, 2000. 17(1): p. 1-17.
83. Zhao, P., S. G. Waxman, and B. C. Hains, Extracellular signal-regulated kinase-regulated microglia-neuron signaling by prostaglandin E2 contributes to pain after spinal cord injury. J Neurosci, 2007. 27(9): p. 2357-68.
84. Corder, G., et al., Constitutive mu-opioid receptor activity leads to long-term endogenous analgesia and dependence. Science, 2013. 341(6152): p. 1394-9.
85. Solway, B., et al., Tonic inhibition of chronic pain by neuropeptide Y. Proc Natl Acad Sci USA, 2011. 108(17): p. 7224-9.
86. Griggs, R. B., M. T. Bardo, and B. K. Taylor, Gabapentin alleviates affective pain after traumatic nerve injury. Neuroreport, 2015. 26(9): p. 522-7.
87. Yezierski, R. P., et al., Spinal neurons involved in the generation of at-level pain following spinal injury in the rat. Neurosci Lett, 2004. 361(1-3): p. 232-6.
88. McKellar, Q. A., et al., Fenbendazole pharmacokinetics, metabolism, and potentiation in horses. Drug Metab Dispos, 2002. 30(11): p. 1230-9.
89. Rogers C, Iqbal H, Davis K, Young S, and Yu C G. Flubendazole reduces α-tubulin acetylation, endosomal signaling, and pain behaviors after excitotoxic spinal cord injury in rats. Abstract, J Neurotrauma. NeuroTrauma-2018, The 3rd Joint Symposium of the International and National Neurotrauma Societies and AANS/CNS Section in Toronto, Canada.
90. Kumar V, Shankaraiah M M, Venkatesh J S, Ranagaraju D, and Nagesh C. Characterization of ternary system of poorly soluble drug in various hydrophilic carriers. International Research Journal of Phamacy, 2011, 2(9): 143-145.
91. Canova, K., L. Rozkydalova, and E. Rudolf, Anthelmintic Flubendazole and Its Potential Use in Anticancer Therapy. Acta Medica (Hradec Kralove), 2017. 60(1): p. 5-11

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of treating pain in a patient having a traumatic spinal cord injury, comprising:
administering an effective amount of flubendazole, an α-tubulin acetylation inhibitor, an endosomal NR1 and pERK1/2 inhibitor, a mitochondrial cyclin b1 inhibitor, a microtubule destabilizing drug, or combinations thereof to the patient.

2. The method of claim 1, wherein the pain is neuropathic pain.

3. The method of claim 1, wherein the pain is caused by excitotoxic neural injury.

4. The method of claim 1, wherein the patient is at risk for developing neuropathic pain.

5. The method of claim 4, comprising administering an effective amount of flubendazole to the patient.

6. The method of claim 5, wherein the treatment comprises substantially preventing neuropathic pain in the patient.
